(19) United States
(12) Patent Application Publication
(10) Pub. No.: US 2004/0186510 A1
(43) Pub. Date: Sep. 23, 2004

(54) Embolic protection ivc filter

(75) Inventor: Karla Weaver, Framingham, MA (US)

Correspondence Address: CROMPTON, SEAGER & TUFTE, LLC, 1221 NICOLLET AVENUE, SUITE 800, MINNEAPOLIS, MN, 55403-2420, US

(73) Assignee: SCIMED LIFE SYSTEMS, INC.

(21) Appl. No.: 10/391,408

(22) Filed: Mar. 18, 2003

(51) Int. Cl. A61M 29/00

(52) U.S. Cl. 606/200

(57) ABSTRACT

Intravascular filter devices adapted for temporary or permanent insertion in a blood vessel are disclosed. An intravascular filter device may comprise a first set of filter legs configured to attach to a vessel wall, and a second set of shorter filter legs configured to attach at a different location to the vessel wall. Both sets of filter legs may include a proximal filtering portion and a distal centering portion, and can be configured to radially expand in an outward direction and with equal force when placed in the vessel. Moreover, the filter device may employ one or more features to ensure uniform clot capturability across the vessel.

### FIELD OF THE INVENTION

The present invention relates to devices for filtering blood clots within a vessel. More specifically, the present invention pertains to filters temporarily or permanently implantable within the vena cava.

### BACKGROUND OF THE INVENTION

Vena cava filters are typically used in combination with other thrombolytic agents to treat pulmonary embolism within a patient. These devices are generally implanted within a vessel such as the inferior vena cava, and function by capturing blood clots (emboli) contained in the blood stream before they can reach the lungs and cause permanent damage to the patient. To trap the emboli, many conventional vena cava filters utilize a plurality of elongated filter legs that can be expanded within the body to form a conical-shaped surface that captures blood clots without disturbing the flow of blood. Once captured, a natural clot lysing process occurs within the body to dissolve the blood clots collected by the filter.

Delivery of the vena cava filter within the body is generally accomplished via an introducer catheter or sheath percutaneously inserted through the femoral (groin) or jugular (neck) veins. Such introducer catheters or sheaths are generally tubular in shape, and include an inner lumen configured to transport the filter in a collapsed position through the body. Once transported to a desired location in the body (e.g. the inferior vena cava), the filter can then be removed from within the catheter or sheath, allowing the filter legs to spring open and engage the vessel wall. A hook, barb or other piercing means disposed on each filter leg can be used to secure the filter to the vessel wall.

### SUMMARY OF THE INVENTION

The present invention relates to devices for filtering blood clots within a vessel. In an exemplary embodiment of the present invention, an intravascular filter device may comprise a first set of elongated filter legs configured to engage the vessel wall at a first location, and a second set of elongated filter legs configured to engage the vessel wall at a second location longitudinally spaced from the first location. Each of the first and second sets of filter legs may include a proximal filtering portion configured to capture blood clots contained in the blood, and a distal centering portion configured to center the filter within the vessel.

The proximal filtering portion of each filter leg may include one or more zigzag regions which, when expanded in the vessel, increase the total surface area of the filter. The size and shape of the zigzag regions can be selected to impart a particular degree of clot capturability without disrupting the flow of blood through the vessel. In certain embodiments, the dimensions of the first and/or second set of filter legs may be selected to provide uniform clot capturability across the vessel.

The distal centering portion of each filter leg may include a hook region configured to either temporarily or permanently engage the vessel wall. The hook region may vary in dimensions, and may be configured to disengage from the wall of the vessel with minimal trauma, leaving only a ring of contact points about the vessel wall. The distal centering portion of each filter leg may be tapered in the distal direction to reduce the profile of the intravascular filter when loaded into an introducer sheath. The base diameters formed by the first and second set of filter legs can be adjusted to compensate for the loss of radial force exerted on the vessel wall resulting from the taper.

### BRIEF DESCRIPTION OF THE DRAWINGS

FIG. 1 is a perspective view of an intravascular filter device in accordance with an exemplary embodiment of the present invention;

FIG. 2 is a top view of the intravascular filter of FIG. 1;

FIG. 3 is a diagrammatic view of the intravascular filter of FIG. 1, showing the filter deployed within a blood vessel;

FIG. 4 is an exploded view of the distal portion of a filter leg from the first set of filter legs, showing the filter leg engaged along the vessel wall;

FIG. 5 is an exploded view of the distal portion of a filter leg from the second set of filter legs, showing the filter leg engage along the vessel wall;

FIG. 6 is a perspective view of an intravascular filter device in accordance with another exemplary embodiment of the present invention;

FIG. 7 is an exploded view of the distal portion of a filter leg from the second set of filter legs, showing the filter leg engaged along the vessel wall;

FIG. 8 is a perspective view of an intravascular filter device in accordance with yet another exemplary embodiment of the present invention; and

FIG. 9 is an exploded view of the distal portion of a filter leg from the second set of filter legs, showing the filter leg engaged along the vessel wall.

### DETAILED DESCRIPTION OF THE INVENTION

The following description should be read with reference to the drawings, in which like elements in different drawings are numbered in like fashion. The drawings, which are not necessarily to scale, depict selected embodiments and are not intended to limit the scope of the invention. Although examples of construction, dimensions, and materials are illustrated for the various elements, those skilled in the art will recognize that many of the examples provided have suitable alternatives that may be utilized.

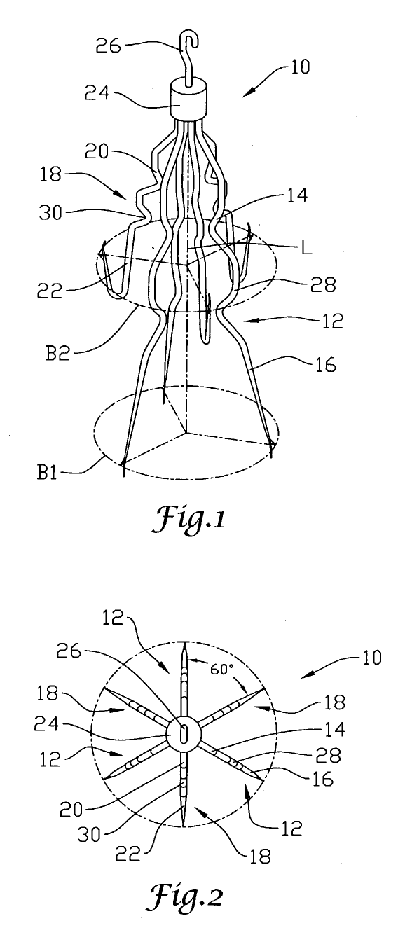

FIG. 1 is a perspective view of an intravascular filter 10 in accordance with an exemplary embodiment of the present invention. Intravascular filter 10 comprises a first set of elongated filter legs 12 each having a proximal filtering portion 14 and a distal centering portion 16, and a second set of elongated filter legs 18 of shorter length each having a proximal filtering portion 20 and a distal centering portion 22. The first and second sets of elongated filter legs 12,18 are each attached proximally to an apical head 24 forming an apex of the filter 10. As is discussed in greater detail below, each set of filter legs 12,18 are configured to radially expand and engage the inner wall of a blood vessel at two different longitudinally spaced locations.

The apical head 24 defines a common longitudinal axis L about which both sets of elongated filter legs 12,18 are configured to expand symmetrically when deployed in the blood vessel. The elongated filter legs 12,18 can be bonded to the apical head 24 by any number of suitable bonding techniques, including soldering, crimping, welding (e.g. laser spot welding) or adhesion. A hook 26 or other retrieval means may be attached to the apical head 24 for retrieving the filter 10 from the blood vessel.

The first set of filter legs 12 may be formed from a metal or metal alloy such as titanium (e.g. Beta III titanium), platinum, stainless steel (e.g. type 304 or 316), or cobalt-chrome alloy. Each filter leg 12 may be formed of round or rectangular shaped wire stiff enough to retain the generally conical shape of the filter, yet small enough to fit within the introducer catheter or sheath. In certain embodiments, for example, the wire may have a circular cross-section having a diameter between 0.016 inches and 0.020 inches. More specifically, the filter legs 12 may have a diameter of 0.016 inches, thus reducing the profile of the device when loaded into the introducer catheter or sheath. The filter legs 12 may also be coated with a layer of an anti-thrombogenic material such as heparin (or its derivatives), urokinase, or PPack (dextrophenylalanine proline arginine chloromethylketone) to prevent insertion site thrombosis from occurring within the vessel.

In certain embodiments, the first set of filter legs 12 may comprise a material having superelastic and/or shape-memory characteristics such as nickel-titanium alloy (Nitinol). A slight outward bend can be imparted to each filter leg 12 by heating the alloy beyond its final austenitic temperature, and then bending each filter leg 12 to a pre-defined shape. The filter legs 12 can be configured to revert to their pre-defined (i.e. bent) shape at or near body temperature such that the filter legs 12 remain straight until deployed in the vessel.

The proximal filtering portion 14 of each filter leg 12 diverges from the apical head 24 at an angle to form a generally conical-shaped surface upon which emboli contained in the blood vessel is collected. The filter legs 12 can be dimensioned identically with respect to each other such that, when intravascular filter 10 is deployed within the vessel, each of the filter legs 12 engage the cava wall at the same longitudinal location within the vessel. In addition, each of the filter legs 12 can be spaced circumferentially at equidistant intervals such that the intravascular filter 10 symmetrically engages the vessel wall.

The proximal filter portion 14 of each filter leg 12 may include one or more zigzag regions 28 along each filter leg 12. The zigzag regions 28 function by increasing the total surface area along the proximal filtering portion 14 of the filter 10. The size and shape of the one or more zigzag regions 28 can be selected to provide a particular degree of clot capturability within the vessel while maintaining the flow of blood through the filter 10. In those embodiments employing a shape-memory material, the zigzag regions 28 can be configured to revert from a straight shape to the zigzag shape when deployed in the vessel, reducing the profile of the filter legs 12 when loaded into the introducer catheter or sheath.

The second set of elongated filter legs 18 may be configured similar to the first set of elongated filter legs 14, but are generally shorter in length. In the exemplary embodiment illustrated in FIG. 1, the second set of filter legs 18 are configured to impart the same radial force along the vessel wall as the first (i.e. longer) set of filter legs 12.

The amount or radial force each of the filter legs 12,18 exerts on the inner wall of the vessel is dependent on several factors, including the diameter of the wire, and the nominal (static) base diameter of the filter. To compensate for the shortened length, the second set of filter legs 18 may diverge from the apical head 24 at a greater angle relative to the longitudinal axis L of the filter 10 such that the base diameter B₂ of the second set of filter legs 18 is similar or equal to the base diameter B₁of the first set of filter legs 12.

Each of the second set of filter legs 18 may also include one or more zigzag regions 30 along their length, which, as discussed above, provide additional surface area to capture blood clots contained in the blood stream. To compensate for the shortened length of the filter legs 18, the size and shape of the one or more zigzag regions 30 can be adjusted such that the surface area of the second set of filter legs 18 is similar or equal to the surface area of the first set of filter legs 12, thus providing uniform clot capturability across the vessel.

FIG. 2 is a top perspective view of the intravascular filter 10 of FIG. 1. As shown in FIG. 2, each filter leg 12,18 may be disposed at equidistant intervals (i.e. 60°) intervals with respect to each other. In addition, the filter legs 12,18 can be arranged in alternating fashion such that each filter leg from the first set of filter legs 12 is located radially adjacent a filter leg from the second set of filter legs 18.

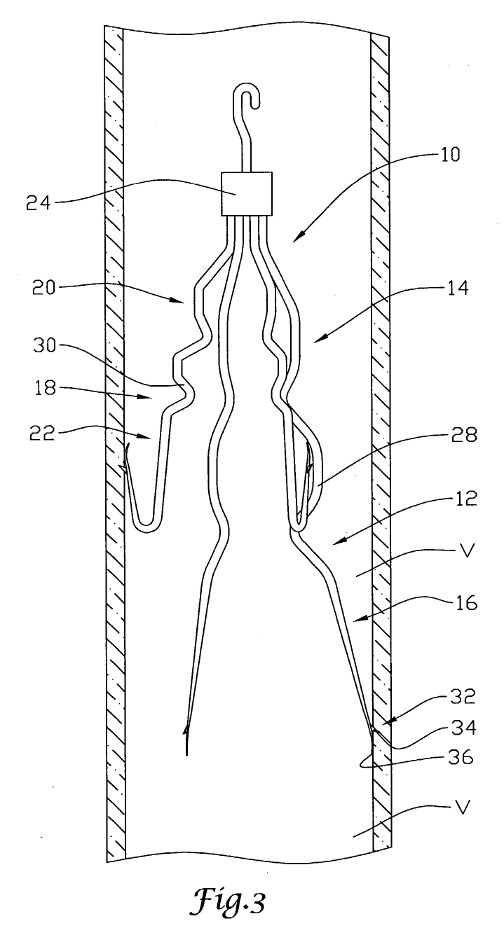

FIG. 3 is a diagrammatic view of intravascular filter 10, showing the filter deployed within a blood vessel V. As shown in FIG. 3, the first and second set of filter legs 12,18 are each configured to engage the vessel wall at different longitudinal locations, providing two levels of contact within the vessel V. In use, the filter legs 12,18 act to resist tilting and to center the intravascular filter 10 within the vessel V. The filter legs 12,18 are also configured to hold the intravascular filter 10 in place to prevent migration towards the heart or legs. The ability of the intravascular filter 10 to self-center upon insertion allows the device to be inserted in a wide range of lumen configurations with different placement techniques.

The distal centering portion 16 of the first set of filter legs 12 may include a hook region 32 configured to releasably secure the intravascular filter 10 to the vessel wall. The hook region 32 may comprise a distally facing hook 34 adapted to pierce the vessel wall. The hook 34 may be formed from the same piece of wire as that used to form the remaining portion of the filter leg 12, or may be formed as a separate element and attached to the distal centering portion 16 of each filter leg 12. In the latter case, the hook 34 can be attached to the filter leg 12 via a laser welding, soldering, crimping, adhesion or other suitable process. Each hook 34 can be configured to disengage from the vessel wall in the same direction as inserted, leaving only a ring of contact points. The distal end 36 of each filter leg 12 may have a slight inward bend to prevent it from piercing the cava wall.

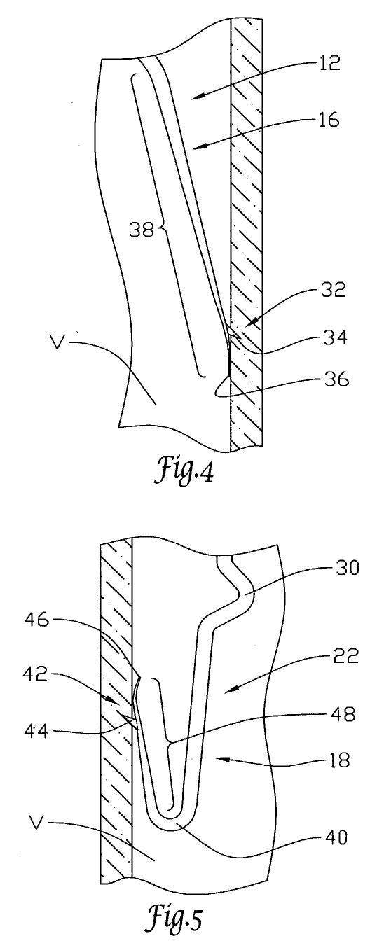

The distal centering portion 16 of one or more of the first set of filter legs 12 may be tapered in the distal direction to reduce the profile of the intravascular filter 10. As shown in FIG. 4, for example, the elongated filter leg 12 may taper along a length 38 from a generally 0.016 inch diameter to a 0.001 inch diameter at the distal end 36 of the filter leg 12.

To compensate for the loss in radial force exerted on the wall due to the decrease along tapered length 38, the base diameter of the first set of filter legs 12 can be increased. In certain embodiments, for example, the base diameter of the first set of filter legs 12 can be increased from 38.5 mm to 55 mm to enable ligation within a vessel having a size in the range of 14 mm to 28 mm. It should be understood, however, that the necessary expansion in the base diameter is dependent on many factors, including the dimensions and composition of the filter legs, and the dimensions of the vessel. As such, the necessary change in the base diameter may vary depending on the particular application.

FIG. 5 is a perspective view showing the distal centering portion 22 of one of the second set of filter legs 18. As shown in FIG. 5, distal centering portion 22 extends distally from the zigzag region 30, and is bent back proximally a short distance about a bend region 40. As with the first set of filter legs 12, a hook region 42 on each filter leg 18 is configured to pierce the vessel wall. The hook region 32 may comprise a proximally facing hook 44 configured to pierce the vessel wall, forming a second ring of contact points about the vessel V. In addition, the hook 44 can be configured to disengage from the vessel wall in the same direction as engaged to facilitate removal of the intravascular filter 10 from the body, if desired. The end 46 of each filter leg may have a slight inward bend to prevent the end 46 from piercing the vessel wall.

In some embodiments, the distal centering portion 22 can be tapered to permit the hook region 42 to bend and flex about the bend region 40. For example, the distal centering portion 22 may taper along a length 48 from a generally 0.016 inch diameter at the bend region 40 to a 0.001 inch diameter at the distal end 46 of the filter leg 18. The reduction of diameter along length 48 allows the filter legs 18 to fold radially inwardly within the introducer catheter or sheath.

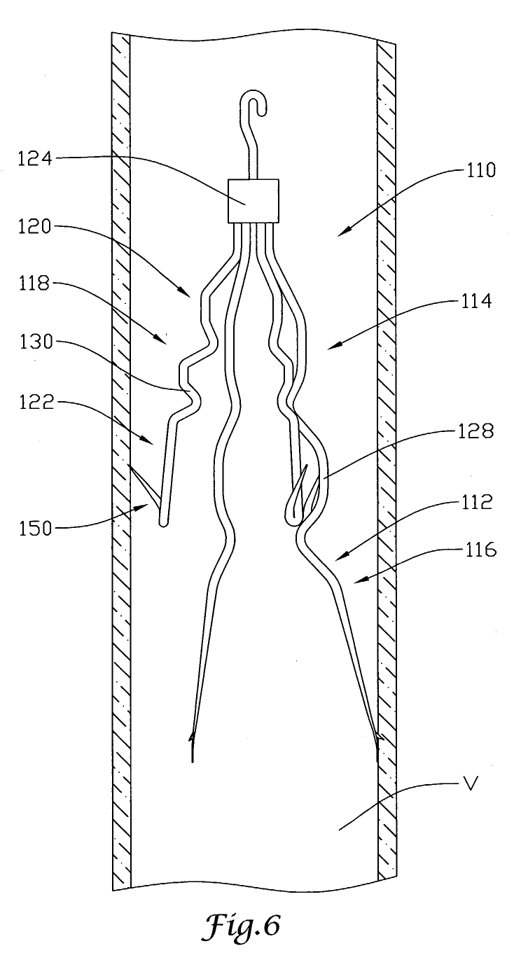

Referring to FIG. 6, an intravascular filter 110 in accordance with another exemplary embodiment of the present invention will now be described. Intravascular filter 110 comprises a first set of elongated filter legs 112 each having a proximal filtering portion 114 and a distal centering portion 116, and a second set of elongated filter legs 118 of shorter length each having a proximal filtering portion 120 and a distal centering portion 122. As with intravascular filter 10, the first and second sets of elongated filter legs 112,118 are each attached proximally to an apical head 124, and are configured to radially expand and engage the inner wall of a blood vessel V at two different longitudinally spaced locations, providing two levels of contact within the vessel V.

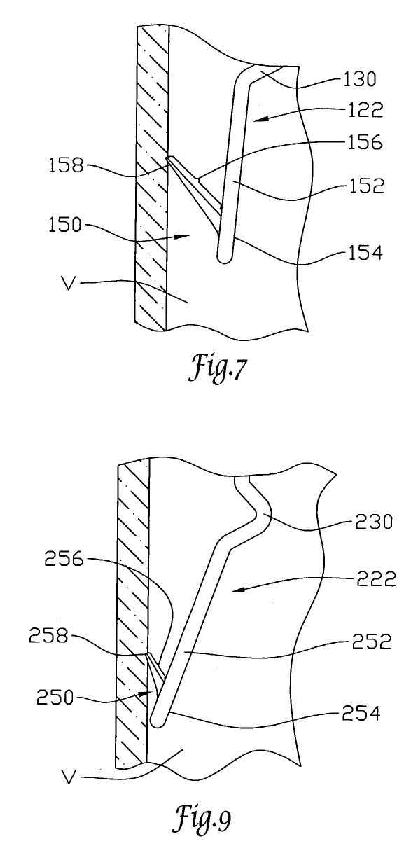

In the exemplary embodiment illustrated in FIG. 6, the distal centering portion 122 of the second set of filter legs 118 may include a reversibly bent hook region 150 configured to engage the vessel wall. As shown in greater detail in FIG. 7, hook region 150 comprises a main section 152, and a reversibly bent section 154 bent through an angle of about 180° in the plane tangential to the conical configuration of the leg 118 and disposed parallel and contiguous to the main section 152. Extending further, and at an angle relative to the reversibly bent portion 154, the hook region 150 tapers along a length 156 to a pointed tip 158 at the end of the filter leg 118.

In some embodiments, the length 156 may taper from a generally 0.016 inch diameter to a 0.001 inch diameter at the end 158 of the filter leg 118. In use, the taper imparts flexibility to the filter legs 118, providing enough radial force to prevent migration of the intravascular filter 10, but having a sufficient amount of resiliency to permit the filter legs 118 to be compressed into the introducer catheter or sheath and regain their original shape after being released in the vessel V.

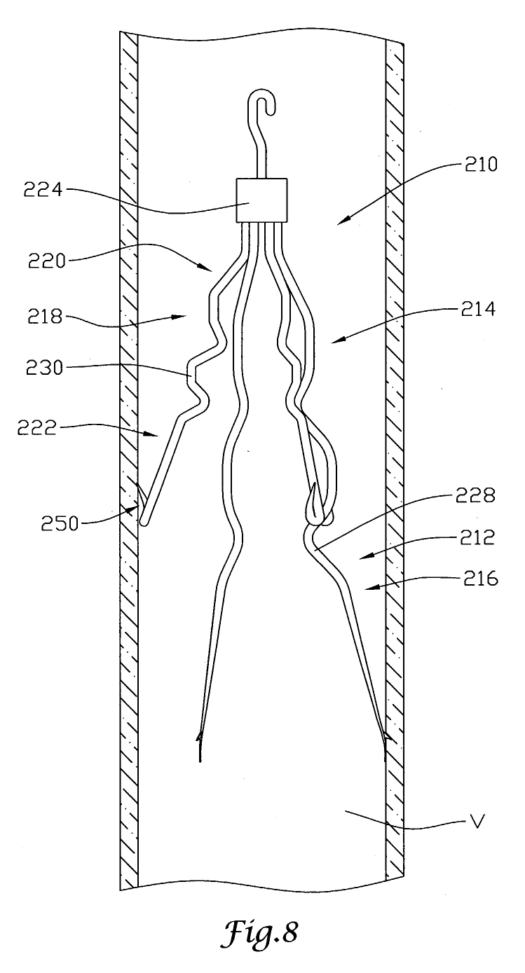

The length of the taper can also be selected such that the base diameter of the second set of filter legs 118 is similar or equivalent to the base diameter of the first set of filter legs 112. In an alternative embodiment shown in FIGS. 8 and 9, for example, the length 256 of the taper may be relatively small in comparison to length 156 illustrated in FIGS. 6 and 7. To compensate for the shorter length, and to ensure that the base diameters of the first and second sets of filter legs 212,218 are equivalent, the second set of filter legs 218 may diverge from the apical head 224 at a greater angle relative to the longitudinal axis of the intravascular filter 10. In addition, the zigzag region 230 of intravascular filter 210 can be increased in size to compensate for the shorter length of the second set of filter legs 218 such that both sets of filter legs 212,218 have approximately the same surface area to ensure uniform clot capturability across the vessel V.

Having thus described the several embodiments of the present invention, those of skill in the art will readily appreciate that other embodiments may be made and used which fall within the scope of the claims attached hereto. Numerous advantages of the invention covered by this document have been set forth in the foregoing description. It will be understood that this disclosure is, in many respects, only illustrative. Changes may be made in details, particularly in matters of shape, size and arrangement of parts without exceeding the scope of the invention.

What is claimed is:

1. An intravascular filter device, comprising:
a first plurality of elongated filter legs each having a proximal filtering portion and a distal centering portion, the distal centering portion of said first plurality of elongated filter legs tapering along a portion thereof; and
a second plurality of elongated filter legs each having a proximal filtering portion and a distal centering portion, the distal centering portion of said second plurality of elongated filter legs tapering along a portion thereof.

2. The intravascular filter device of claim 1, further comprising an apical head.

3. The intravascular filter device of claim 2, wherein said apical head includes a retrieval hook.

4. The intravascular filter device of claim 1, wherein said first and second plurality of elongated filter legs are formed of a metal.

5. The intravascular filter device of claim 4, wherein said metal is Beta III titanium.

6. The intravascular filter device of claim 4, wherein said metal is superelastic.

7. The intravascular filter device of claim 4, wherein said metal is a shape-memory metal.

8. The intravascular filter device of claim 1, wherein each of said first and second plurality of elongated filter legs are formed of round wire having an outer diameter of about 0.016 inches.

9. The intravascular filter device of claim 1, wherein the length of said second plurality of elongated filter legs is shorter than the length of said first plurality of elongated filter legs.

10. The intravascular filter device of claim 1, wherein each of said first and second plurality of elongated filter legs includes at least one zigzag region.

11. The intravascular filter device of claim 10, wherein the proximal filtering portion of said first plurality of elongated filter legs has a surface area substantially equivalent to that of said second plurality of elongated filter legs.

12. The intravascular filter device of claim 1, wherein each of said first and second plurality of elongated filter legs includes a hook region adapted to releasably secure to a vessel wall.

13. The intravascular filter device of claim 12, wherein the hook region on said second plurality of elongated filter legs comprises a main section, a reversibly bent portion, and a pointed tip section.

14. The intravascular filter device of claim 1, wherein each of said first and second plurality of elongated filter legs defines a base diameter.

15. The intravascular filter device of claim 14, wherein the base diameters of said first and second plurality of elongated filter legs are substantially equivalent.

16. An intravascular filter device, comprising:
a first plurality of elongated filter legs each having a proximal filtering portion and a distal centering portion, the distal centering portion of said first plurality of elongated filter legs tapering along a portion thereof and including a hook region adapted to releasably secure to a vessel wall; and
a second plurality of elongated filter legs each having a proximal filtering portion and a distal centering portion, the distal centering portion of said second plurality of elongated filter legs tapering along a portion thereof and including a hook region adapted to releasably secure to the vessel wall proximal said first plurality of elongated filter legs.

17. The intravascular filter device of claim 16, further comprising an apical head.

18. The intravascular filter device of claim 17, wherein said apical head includes a retrieval hook.

19. The intravascular filter device of claim 16, wherein said first and second plurality of elongated filter legs are formed of a metal.

20. The intravascular filter device of claim 19, wherein said metal is Beta III titanium.

21. The intravascular filter device of claim 19, wherein said metal is superelastic.

22. The intravascular filter device of claim 19, wherein said metal is a shape-memory metal.

23. The intravascular filter device of claim 16, wherein each of said first and second plurality of elongated filter legs are formed of round wire having an outer diameter of about 0.016 inches.

24. The intravascular filter device of claim 16, wherein the length of said second plurality of elongated filter legs is shorter than the length of said first plurality of elongated filter legs.

25. The intravascular filter device of claim 16, wherein each of said first and second plurality of elongated filter legs includes at least one zigzag region.

26. The intravascular filter device of claim 25, wherein the proximal filtering portion of said first plurality of elongated filter legs has a surface area substantially equivalent to that of said second plurality of elongated filter legs.

27. The intravascular filter device of claim 16, wherein the hook region on said second plurality of elongated filter legs comprises a main section, a reversibly bent portion, and a pointed tip section.

28. The intravascular filter device of claim 16, wherein each of said first and second plurality of elongated filter legs defines a base diameter.

29. The intravascular filter device of claim 28, wherein the base diameters of said first and second plurality of elongated filter legs are substantially equivalent.

30. An intravascular filter device, comprising:
a first plurality of elongated filter legs each having a proximal filtering portion and a distal centering portion, the distal centering portion of said first plurality of elongated filter legs tapering along a portion thereof and including a distally facing hook adapted to releasably secure to a vessel wall; and
a second plurality of elongated filter legs each having a proximal filtering portion and a distal centering portion, the distal centering portion of said second plurality of elongated filter legs tapering along a portion thereof and including a proximally facing hook adapted to releasably secure to the vessel wall.

31. An intravascular filter device, comprising:
an apical head defining a central longitudinal axis;
a first plurality of elongated filter legs radially expandable in an outward direction from the longitudinal axis, each of said first plurality of elongated filter legs being secured to the apical head and having a proximal filtering portion and a distal centering portion, the proximal filtering portion of said first plurality of elongated filter legs including at least one zigzag region, the distal centering portion of said first plurality of elongated filter legs tapering along a portion thereof and including a distally facing hook adapted to releasably secure to a vessel wall; and
a second plurality of elongated filter legs radially expandable in an outward direction from the longitudinal axis, each of said second plurality of elongated filter legs being secured to the apical head and having a proximal filtering portion and a distal centering portion, the proximal filtering portion of said first plurality of elongated filter legs including at least one zigzag region, the distal centering portion of said second plurality of elongated filter legs tapering along a portion thereof and including a proximally facing hook adapted to releasably secure to the vessel wall.
